(19) United States
(12) Patent Application Publication
(10) Pub. No.: US 2023/0143619 A1
(43) Pub. Date: May 11, 2023

(54) Method for predicting miscarriage and iInfertility before or after conception - us bi patent #6,347,634 as of reference

(71) Applicant: Francis Jancik, Anaheim, CA (US)

(72) Inventor: Francis Jancik, Anaheim, CA (US)

(21) Appl. No.: 17/300,735

(22) Filed: Nov. 8, 2021

(51) Int. Cl. A61B 5/00; A61B 5/01; G01J 5/00

(52) U.S. Cl. CPC A61B 5/4343; A61B 5/01; A61B 5/4375; A61B 5/743; G01J 5/0025

(57) ABSTRACT

A method of increasing the probability that a woman and/or a father will conceive either INFERTILITY or MISCARRIAGE before or after conception, comprising the steps of determining an infrared body temperature of the mother, determining an infrared body temperature of the father, categorizing the determined infrared body temperatures of the mother and the father as either “very extremely low” (Infertility) or “very extremely high” (Miscarriage) according to a predetermined categorization criteria, and correlating the stress level of the mother and the father according to a correlation where in the case of very extremely low temperature for either or both the mother and the father, the probability of INFERTILITY, according to a predetermined categorization criteria, and correlating the stress level of the mother and the father according to a correlation where in the case of very extremely high temperature for either or both the mother and the father, the probability of MISCARRIAGE. The information derived is applied by using sex probabilities according to the criteria where procuring conception of the “stillborn child” (Miscarriage) or “not exited” (Infertility) without alteration of the infrared body temperature of the mother and father if the indicated probability of not conceiving a child, or procuring conception of stillborn baby after alerting the infrared body temperatures of mother and/or father to increase the indicated probability of not conceiving an alive child of either of

This application is a continuation of Ser. No. 17/300,735, filed Jun. 25, 2021

### TECHNICAL FIELD AND BACKGROUND OF THE INVENTION

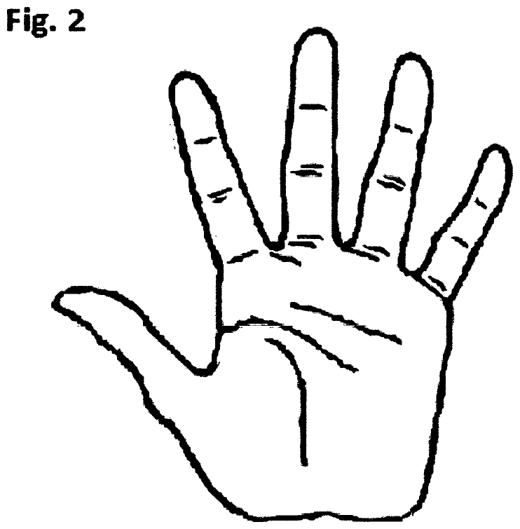

This invention relates to a method of warning the probability of a female parent losing a fetus of less 6 weeks or a pregnancy loss within 20 weeks, by of infrared radiation analysis to techniques involve manipulating the father's sperm to either a male offspring or miscarriage exposed to the mother's egg for fertilization or non-fertilization depending on determining an infrared body temperature of father and/or mother showing “extremely high” above 35.7 degrees Celsius or almost full red infrared image (See FIG. 2) to show overheat scale of miscarriage before or after conception by Infrared imaging IRISYS Laser Camera connected to Palm Hand Scale Temperature Ranges to measure how much heat in an infrared body temperature of father and/or mother. (See FIG. 2).

This method has application by medical personnel during individual counseling of couples being warned to increase the probability of having a risk of losing a baby before conception or after pregnancy in advance of possible saving a baby within a limited time at a slightly chance. Successfully a baby was saved from almost miscarriage with the help of infrared radiation analysis to warn a female parent to decrease the overheat stress in her body in less 20 weeks.

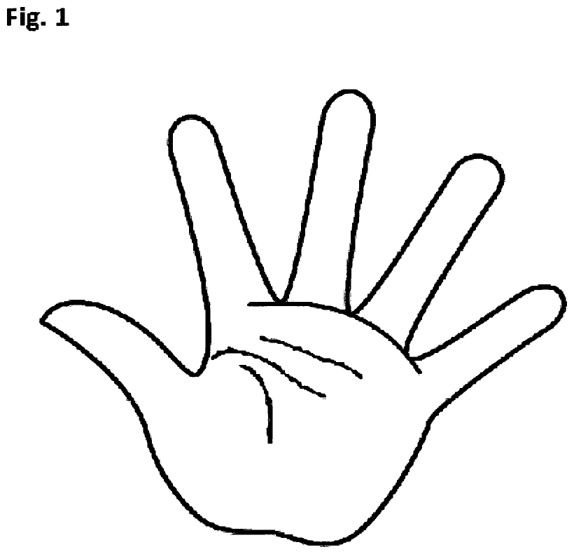

For Infertility warnings by use of infrared radiation analysis to measure below or “extremely low” below 29 degrees Celsius or almost 100% black infrared image (See FIG. 1) for either the mother or father for high risk of infertility, they will know there is of no chance to have a baby in advance rather than not knowing. Infertility shows no color (black) or very tiny color spot on black photo (See FIG. 1) by no chance of having a baby forever according to infrared radiation analysis.

One couple from Maryland spent over $15,000 at a 3-day hospital performed by a special doctor to hope to have a baby but still infertility to damage their finances, emotions and perhaps marriage problem with no knowledge.

This method has application by medical personnel during individual counseling of couples knowing which one of the probability of having either miscarriage before or after conception or infertility before conception by use of infrared radiation analysis, as well as by government officials as a part of population control practices. Use of the technique described in this application will substantially reduce pains, financial losses and others.

Also counseling the couples which one of girl (already patented of preconception) or boy (already patented of preconception) of the probability of having what gender of future child before conception. (See FIG. 3).

Techniques for enhancing the probability of saving a boy baby of determined sex are known by warning a possible miscarriage before or after conception to reduce a stress early before the result happened at low percentage of better chance to save a baby. In general, these techniques involve manipulating the father's sperm to either a male offspring or miscarriage exposed to the mother's egg for fertilization or non-fertilization depending on determining an infrared body temperature of father and/or mother. (See FIG. 3).

A method of predicting whether a conceiving couple are at high risk of miscarriage before or after pregnancy or infertility before pregnancy, comprising the steps of: (a) determining an infrared body temperature of the father using data obtained by a medical thermograph; (b) determining an infrared body temperature of the mother using data obtained by the medical thermograph; (c) categorizing the determined infrared body temperatures of the mother and the father as either “extremely high” (above 35.7 degrees Celsius) or “extremely low” (below 29 degrees Celsius); (d) correlating the infrared body temperatures of the mother and the father according to the categorization criteria wherein: (i) in the case of high temperature for the mother and/or the father, the probability of a miscarriage is very high; (ii) in the case of high temperature for one of the mother or the father and low temperature for the other of the mother or the father, the probability of a baby or miscarriage stands at 50 percent chance; and (iii) in the case of low temperature for either the mother and/or the father, the probability of infertility stands at greater than 80% chance.

This invention relates to a method of increasing the probability of predicting the likelihood of miscarriage or infertility, by use of infrared radiation analysis. This method has application for personnel during individual counseling of couples wishing to avoid a miscarriage or problems of infertility and increase the probability of having either a boy or a girl (U.S. Pat. No. 6,347,634 B1 as of Reference), as well as by government officials as a part of population control practices. Use of the technique described in this application will substantially reduce miscarriages under 34.9 degrees Celsius. The modification adds that readings above 35.0 degrees Celsius are at high risk for a miscarriage and readings below 29 degrees Celsius or almost 100% black infrared image (See FIG. 1) are at a high risk of being infertile.

A Miscarriage before or after Conception is recommended to be tested with the infrared device within 30-45 days on

both father and mother. A Miscarriage usually happens in the first 90 days of pregnancy to be required to have an infrared test within 30-45 days from the first day of pregnancy to be warned in time of a treatment possibly. For Infertility, there is no limit time—just warning before conception attempt.

Presently Using IRISYS Universal Thermal Imager with Laser Radiation instead of old Inframetrics Model 535 for using for girl and boy perconception prior to getting patented. The Infrared Imager is a more cost efficient way to scan. Unlike the 535 Model, the IRISYS does not require liquid nitrogen which runs on batteries. The IRISYS can scan any surface and produce thermal readings from −10 Degrees to +300 Degrees Celsius.

The ergonomically designed imager houses the imaging optics, detector; drive electronics, optical modulator, laser pointer and four standard or rechargeable AA size batteries or plug unit. The system includes an optional pistol grip

handle, which holds virtually any ‘Pocket PC’ or compatible ‘Palm’ device as a combined processing, display unit and image storage device. As an alternative, the output of the imager can be displayed and processed in real time using a PC.

Infrared analysis of human beings has been common for many years. Many medical procedures, such as determining blood circulation efficiency and the location of tumors, rely on thermographs taken of parts of the human body. Human skin is an almost perfect absorber, and hence, emitter of infrared radiation. With a perfect emitter of infrared radiation defined as having an emissive index of 1.00, human skin has an emissive index of 0.98. This means infrared radiometric techniques are relatively simple and accurate when used in measuring the temperature of human skin.

Infrared temperature measurement is carried out with an infrared scanning device. The scanning field is optically scanned point by point to create a mosaic picture. This happens at a scan rate sufficiently rapid that the image generated appears instantaneous and continuous, as in PALM HAND device connected to IRISYS Thermal Imager Camera—FDA-Approval. A typical infrared scanner acquires infrared radiation from the target and reflects the radiation onto a vertical oscillating mirror, which reflects it upwardly to a horizontally oscillating mirror. From the horizontal mirror the radiation is reflected downwardly to an angled mirror, which sends the beam of laser radiation to the lens to record on PALM HAND device like Cell Phone, which focuses it onto the detector. The infrared beam is converted to an electrical signal, which is amplified and processed electronically into a PALM-type picture.

In general, the method according to the invention operates on the principle that there is a relatively high correlation between the infrared body temperature of human beings and the effect of that temperature on the probability of either a X-chromosome (female gender determining) or Y-chromosome (male gender determining) sperm achieving union with the female's egg cell.

While there is much that is not known about the effects of temperature, stress and similar human characteristics on sperm behavior, it is known that human stress levels are reflected in varying infrared body temperatures, and that body temperature has an effect on sperm motility and survival.

Mostly the father tends to have a 50% chance to determine what gender of a baby birth. For Mothers, 80% up of girl favorite predications normally unless it is in her human-hand temperatures that shown with the biological changes due to a hard nature cause. During World Wars, more boy babies were born at high 80% in fact. (Perhaps more miscarriages. My late mother lost a baby by miscarriage in 1943.)

The inventive method according to this application permits a desiring couple to know before conception whether they are at risk of a miscarriage or infertility without expensive and complicated medical procedures and damages of finance, possible marriage, possible abuses, keeping their hopes like unnecessary “too many” boys or girls in one family, emotional injuries, killing unwanted babies in favor of boy baby globally.

Advanced Knowledge is the best to determine what to do. All works by same infrared radiation analysis have been done with the answers in less 5 minutes from one of 4 options: Miscarriage, infertility, girl or boy in less 1 minute average mostly and cheaply. No need for the stay 3-day hospital less accurately and more costly.

(For example: Henry VIII is best known for his 6 wives, & several mistresses he kept on the side. The monarch's desperate quest for political unification and a healthy male heir drove him to annul two marriages & have two wives beheaded. No doubt—the last wife was stressful to birth a boy.)

### SUMMARY OF THE INVENTION—(SEE FIG. 3)

Therefore, it is an object of the invention to provide a method of predicting (before or after conception) the likelihood of a couple suffering a miscarriage or infertility by use of infrared radiation analysis.

It is another object of the invention to provide a method of predicting a successful conception, which can either confirm the parents' ability to reproduce in a normal human manner, or whether they may need medical intervention to avoid miscarriage. Infertility is unavoidable up to today.

It is another object of the invention to provide a method of increasing the probability of predicting miscarriages or infertility, which utilizes information regarding the infrared temperature of the parents.

This is a modification of the invention, U.S. Pat. No. 6,347,634 B1 as of Reference, which serves to provide a method of predicting and modifying the probability of a couple conceiving a child of the desired sex based on normal ranges of body temperature readings as measured by a infrared thermograph. In Northern America, 56% of the parents with their first baby wanted to know what gender of their next baby in advance.

For Miscarriage or Infertility, 99% of the parents wanted to know why that happened. These and other objects of the present invention modification to include the probability of infertility and miscarriage early warnings.

These and other objects of the present invention modification to include the probability of infertility and miscarriage are similar to those achieved in U.S. Pat. No. 6,347,634 B1 as of Reference by providing a method of increasing the predictability of infertility and miscarriage, comprising the steps of determining a baseline stress level of the father, determining a baseline stress level of the mother, categorizing the determined stress levels of the mother and the father as either “extremely high” or “extremely low” according to a predetermined categorization criteria, and correlating the stress level of the mother and the father according to a correlation where in the case of extremely high stress for both the mother and the father, the probability of a miscarriage is very high, with decreased possibilities of miscarriage if the father's readings are below 34.9 degrees Celsius. (See FIG. 3).

In the case that one or both of the conceiving couple displays a reading below 29 degrees Celsius, then the likelihood of infertility and inability to conceive stands at 80 percent up.

According to one preferred embodiment of the invention, the step of determining the stress level of the mother and the father comprises determining the infrared temperature of a body appendage of each.

According to another preferred embodiment of the invention, the body appendages are a mother's normal temperature hand and a father's normal temperature hand. (If the human hands are cold or hot, so it requires waiting till the temperature of their hands has to be normal warmly).

According to yet another preferred embodiment of the invention, the step of procuring conception of the Non-child or child after altering the normal stress level of the mother and/or father includes the step of prescribing medication to reduce the indicated stress level if possibly except incurable Infertility that they know in advance that is better for mental health.

According to yet another preferred embodiment of the invention, the step of procuring conception of the child after altering the normal stress level of the mother and/or father includes the step of prescribing medication to increase the indicated stress level.

According to yet another preferred embodiment of the invention, wherein the steps of determining the stress level of the mother and the father comprise scanning a hand of the mother and a hand of the father with an infrared scanner.

### EVIDENTLY PHOTOS OF INFERTILITY AND MISCARRIAGE

FIG. 1 is a color photograph of a thermogram of a human hand with an isotherm in the “very low stress” zone indicative of a “very low stress” or “black” infrared individual (Infertility).

FIG. 2 is a color photograph of a thermogram of a human left-palm hand with an isotherm in the “very high stress” or “too much red” infrared zone.

(Paul wanted to check if he still could produce a baby but it still showed Infertility in one year later so no matter his mating partner had.) (SEE FIG. 1)

(Maryland woman happened to be infertile to fail to get a pregnancy at several attempts so no matter what her mating partner had. Before I knew her story, she asked me to scan her left palm hand at no charge, it showed there was infertile by 1 minute job to shock her. She explained to me everything about over $15,000 for 3-day work to compare with my work for 1 minute. She cried to break my heart.)

### DESCRIPTION OF THE PREFERRED EMBODIMENT AND BEST MODE

The method according to the preferred embodiment of this invention is carried out using an infrared thermograph, IRISYS Imager computerized medical thermograph manufactured by IRISYS, United Kingdom.

Preferably Other USA-MADE thermographs may also be used.

The method operates on the principle that the human hand can be scanned directly to obtain an indication of stress levels being experienced by the couple. The hand is very rich in blood vessels close to the surface, and thus provides a convenient body appendage from which to obtain a very accurate thermographic picture. The use of the “stress” terminology is only for convenience, and the categorization of a person as having an extremely high or extremely low “stress” level as measured by the thermograph does not mean that the person exhibits traits necessarily associated with stress pathology. The important determinant is the infrared temperature detected by the thermograph.

After proper adjustment of the thermograph according to instructions, the hand of the mother and the father is scanned to determine an isotherm temperature in a normal temperature room. Anisotherm (“ISO”) is a thermal contour line, all points of which are the same temperature. The ISO measurement provides an indication of relative stress level of the subject, according to the following values: (See FIG. 3)

The average High Stress temperature is 34.5° C., and the average Low Stress temperature is 29.2° C. This compares with normal human body core temperature of 37° C., or 98.6° F.

As shown in FIG. 2, Large Area of Red shaded isotherms indicate “very high stress” of Miscarriage, while FIG. 1, Large Area of “Black” isotherms represent “very low stress” areas of Infertility. Thus, the relatively large area of full red shading in FIG. 2 indicates a “very high stress” individual of Miscarriage, while the large areas of light red in FIG. 1 represents a “very low stress” individual of Infertility.

In the case of stress values in the “extremely high” range for both the father and/or mother, the probability of miscarriage is 80% up. In the case of “extremely high” stress readings for the mother and “low” stress readings for the father, the probability of miscarriage or having a male child stands at 50%.

For either the mother or the father not showing any color of blue or red, the probability

Infertility is 80% up. As indicated by a scan to show no sign like almost no color (black), an extremely high chance is infertility.

(Several females had it so far it was 100% accurately I had tested so the males were rare to have the infertility).

Of course, stress levels can change except Infertility, and are subject to alteration for various reasons. Thus, these values may represent long-term traits of an individual or episodic occurrences as a result of externally imposed conditions. These percentages are approximate. It is believed that the range of variation with the “extremely high” and “extremely low” values is between 80% and 99% depending what infrared color is. These probabilities compare favorably with the percentage success rates achieved by currently accepted medical methods. Infrared colors are very important to easily read.

Even if the probabilities are no better than those of currently accepted techniques, the vastly reduced cost and ease of use of the method provide substantial advantages.

Before this invention, there is no known method for predicting miscarriage rates prior to conception and also infertility, too.

With this information, a couple can make an informed decision about whether to attempt conception. If the probabilities favor the normal conception of a child, then conception is attempted. If the probabilities do not favor the normal conception of a child, then the parents may decide not to attempt conception, or may decide to attempt an alteration of the stress levels indicated by the thermograph in order to achieve healthy conception.

While further research must be done in this area, a physician can prescribe various types of stress-modifying medications, or mood-altering techniques such as meditation or alpha-feedback practice may be attempted.

Additional thermographs can easily be taken with little additional expense to determine the effectiveness of these efforts. In any case, these efforts are substantially less invasive and expensive than typical infertility detecting techniques.

A method of increasing the probability of predicting miscarriage (before or after conception) or infertility (before conception) by use of infrared radiation analysis is described above. Various details of the invention may be changed without departing from its scope. Furthermore, the foregoing description of the preferred embodiment of the invention and the best mode for practicing the invention are provided for the purpose of illustration only and not for the purpose of limitation—the invention being defined by the claims. (See FIG. 3)

Extremely High Stress (Large Area of Infrared RED on HUMAN'S LEFT PALM HAND)—Above 35.7° C. (Miscarriage)

Extremely Low Stress (Large Area of Infrared BLACK on HUMAN'S LEFT PALM HAND)—Below 29° C. (Infertility).

Purple Infrared Color—32.2° C. (Either of Girl or Boy).

The average High Stress temperature is 34.1° C. (Boy), and the average Low Stress temperature is 30.4° C.

(Girl). This compares with normal human body core temperature of 37° C., or 98.6° F.

U.S. Pat. No. 6,347,634 B1 (for Girl and Boy only) Issued on Feb. 19, 2002. Estimated Expiration Date: Aug. 16, 2019.

A method of predicting miscarriage (before or after conception) or infertility (before conception) , comprising the steps of determining an infrared body temperature of the father, determining an infrared body temperature of, categorizing the determined infrared body temperatures of the mother and the father as either “extremely high” or “extremely low” according to a predetermined categorization criteria, and correlating the stress level of the mother and the father according to a correlation where in the case of “extremity high” for both the mother and the father, the probability of miscarriage is very high; and in the case of “low” temperature for the father and “extremely high” for the mother, the probability of a male child or miscarriage is more than 50%.

In the case of “extremely low” values for either the mother or the father, the probability of infertility is approximately 80% up accuracy. The derived is applied by using the healthy conception probabilities according to the criteria where procuring healthy conception of the child without alteration of the infrared body temperature of the mother and father if the indicated probability of conceiving naturally, or procuring healthy conception of the child after altering the infrared body temperature of the mother and/or father to increase the indicated probability of healthy conception.

In conclusions, importantly 4 different infrared leveled ranges of Human Left-Palm Hand—Closer to Human Heart:
Infrared Strong Red (Miscarriage at very high chance), Either has it.
Infrared Normal Red (Boy), Once Red is boy—no matter what a mating partner has. Patented.
Infrared Normal Blue (Girl); Both parents must have blue. Patented.
Infrared “Black” (Infertility at very high chance). Once black is Infertility—no matter what a mating partner has.

The probabilities of miscarriage or infertility have been determined empirically from the above information, as follows:

In the case of stress values in the “extremely high” or Large Area of Infrared RED on Human Left-Palm Hand for both the father and/or mother, the probability of miscarriage is 80% up.

In the case of “extremely high” stress readings for the mother and “low” stress readings for the father, the probability of miscarriage stands more than 50%. Always Infrared RED is a boy or not depending how much RED stress is.

Many different models of infrared devices vary in infrared reading measurements but all 2 colored leveled scales of preconception results are a key to determine miscarriage or infertility.

### Evidently Photos of Infertility and Miscarriage

FIG. 1 is a color photograph of a thermogram of a human hand with an isotherm in the “very low stress” zone indicative of a “very low stress” individual (Infertility).

FIG. 2 is a color photograph of a thermogram of a human's left palm hand with an isotherm in the “very high stress” zone

indicative of a “very high stress” individual (Miscarriage); and

(Paul wanted to check if he still could produce a baby but it still showed Infertility in one year later so no matter his mating partner had.)

### DESCRIPTION OF THE PREFERRED EMBODIMENT AND BEST MODE

The method according to the preferred embodiment of this invention is carried out using an infrared thermograph, IRISYS Imager computerized medical thermograph manufactured by IRISYS, United Kingdom.

Preferably Other USA-MADE thermographs may also be used.

The method operates on the principle that the human hand can be scanned directly to obtain an indication of stress levels being experienced by the couple. The hand is very rich in blood vessels close to the surface, and thus provides a convenient body appendage from which to obtain a very accurate thermographic picture. The use of the “stress” terminology is only for convenience, and the categorization of a person as having an extremely high or extremely low “stress” level as measured by the thermograph does not mean that the person exhibits traits necessarily associated with stress pathology. The important determinant is the infrared temperature detected by the thermograph.

After proper adjustment of the thermograph according to instructions, the hand of the mother and the father is scanned to determine an isotherm temperature in a normal temperature room. Anisotherm (“ISO”) is a thermal contour line, all points of which are the same temperature. The ISO measurement provides an indication of relative stress level of the subject, according to the following values: (See Infrared Drawings)

The average High Stress temperature is 34.5° C., and the average Low Stress temperature is 29.2° C. This compares with normal human body core temperature of 37° C., or 98.6° F.

As shown in FIG. 2, Large Area of Red shaded isotherms indicate “very high stress” of Miscarriage, while FIG. 1, Large Area of “Black” isotherms represent “very low stress” areas of Infertility. Thus, the relatively large area of full red shading in FIG. 2 indicates a “very high stress” individual of Miscarriage, while the large areas of barely color of tiny white spot in FIG. 1 represents a “very low stress” individual of Infertility.

In the case of stress values in the “extremely high” range for both the father and/or mother, the probability of miscarriage is 80% up. In the case of “extremely high” stress readings for the mother and “low” stress readings for the father, the probability of miscarriage or having a male child stands at 50%.

For either the mother or the father not showing any color of blue or red, the probability

Infertility is 80% up. As indicated by a scan to show no sign like black, an extremely high chance is infertility.

(Several females had it so far it was 100% accurately so the males are rare to have the infertility).

Of course, stress levels can change except Infertility, and are subject to alteration for various reasons. Thus, these values may represent long-term traits of an individual or episodic occurrences as a result of externally imposed conditions. These percentages are approximate. It is believed that the range of variation with the “extremely high” and “extremely low” values is between 80% and 99% depending what temperature and/or infrared color is. These probabilities compare favorably with the percentage success rates achieved by currently accepted medical methods.

Even if the probabilities are no better than those of currently accepted techniques, the vastly reduced cost and ease of use of the method provide substantial advantages. There is no known method for predicting miscarriage rates prior to conception.

With this information, a couple can make an informed decision about whether to attempt conception. If the probabilities favor the normal conception of a child, then conception is attempted. If the probabilities do not favor the normal conception of a child, then the parents may decide not to attempt conception, or may decide to attempt an alteration of the stress levels indicated by the thermograph in order to achieve healthy conception.

While further research must be done in this area, a physician can prescribe various types of stress-modifying medications, or mood-altering techniques such as meditation or alpha-feedback practice may be attempted. Additional thermographs can easily be taken with little additional expense to determine the effectiveness of these efforts. In any case, these efforts are substantially less invasive and expensive than typical infertility detecting techniques.

A method of increasing the probability of predicting miscarriage or infertility (before or after conception) by use of infrared radiation analysis is described above. Various details of the invention may be changed without departing from its scope. Furthermore, the foregoing description of the preferred embodiment of the invention and the best mode for practicing the invention are provided for the purpose of illustration only and not for the purpose of limitation—the invention being defined by the claims.

1. I, Francis Jancik, claim that a method of predicting infertility before or after conception, comprising the steps of determining an infrared body temperatures of the mother and father, categorizing the determined infrared body temperatures of the mother and the father as either “extremely low” (below 29 Degrees Celsius for risk of infertility—“blank” colorful display on infrared imaging screen).
See FIG. 1
I, Francis Jancik, claim that a method of predicting Miscarriage before or after conception, comprising the steps of determining an infrared body temperature of the mother, categorizing the determined infrared body temperature of the mother “extremely high” (above 35.7 degrees Celsius for risk of miscarriage—more red colorful display on infrared imaging screen).
See FIG. 2
